(19) United States
(12) Patent Application Publication
(10) Pub. No.: US 2018/0171385 A1
(43) Pub. Date: Jun. 21, 2018

(54) ALKB -FACILITATED RNA METHYLATION SEQUENCING (ARM-SEQ)

(71) Applicant: The Regents of the University of California, Oakland, CA (US)

(72) Inventors: Todd LOWE, San Jose, CA (US); Aaron COZEN, Santa Cruz, CA (US); Eva H. ROBINSON, Aptos, CA (US); Andrew D. HOLMES, Santa Cruz, CA (US); Eric PHIZICKY, Pittsford, NY (US); Erin QUARTLEY, Rochester, NY (US)

(73) Assignee: The Regents of the University of California, Oakland, CA (US)

(21) Appl. No.: 15/579,104

(22) PCT Filed: Jun. 2, 2016
(86) PCT No.: PCT/US16/35592

§ 371 Date: Dec. 1, 2017

Related U.S. Application Data
(60) Provisional application No. 62/170,045, filed on Jun. 2, 2015.

(51) Int. Cl. C12Q 1/6806; C12Q 1/6869; C12Q 1/686

(52) U.S. Cl. CPC C12Q 1/6806; C12Q 1/6869; C12Q 1/686; C12Q 2535/122; C12Y 114/11033; C12N 2310/122; C12N 2310/121; C12N 2310/3521; C12Q 2521/125

(57) ABSTRACT

In various embodiments, the invention teaches methods for detecting ribonucleic acid (RNA) molecules that contain certain chemical modifications using sequencing technologies. These modified RNAs are otherwise not readily detected using the commonly used cloning protocols required for sequencing. The method further includes bioinformatics analyses to identify specific RNA species that are modified at high resolution.

### GOVERNMENT RIGHTS

This invention was made with government support under grants HG006753 and GM052347 awarded by the National Institutes of Health. The government has certain rights in the invention.

### FIELD OF THE INVENTION

The present invention generally relates to compositions and methods for nucleotide sequencing.

### BACKGROUND

High throughput RNA sequencing has accelerated discovery of the complex regulatory roles of small RNAs, but RNAs containing modified nucleosides may escape detection when those modifications interfere with reverse transcription during RNA-seq library preparation. There is clearly a need in the art for improved systems and methods for identifying methyl-modified RNAs.

### SUMMARY OF THE INVENTION

A method, including providing a ribonucleic acid (RNA); and applying a quantity of a de-alkylating enzyme to the RNA. In some embodiments, the RNA includes all or a portion of a tRNA. In certain embodiments, the RNA includes one or more of 1-methyladenosine, 3-methylcytidine, and 1-methylguanosine. In some embodiments, the RNA includes one or more of 1-methyladenosine, 3-methylcytidine, and 1-methylguanosine. In certain embodiments, the method further includes sequencing all or a portion of the RNA, thereby determining a post-de-alkylating-enzyme treated RNA sequence. In some embodiments, the de-alkylating enzyme includes Escherichia coli (E. Coli) AlkB.

In various embodiments, the invention teaches a composition that includes a ribonucleic acid (RNA) that has been treated with a de-alkylating enzyme. In some embodiments, the RNA includes tRNA. In certain embodiments, the RNA comprised one or more of 1-methyladenosine, 3-methylcytidine, and 1-methylguanosine prior to treatment with the de-alkylating enzyme. In some embodiments, the RNA includes one or more of 1-methyladenosine, 3-methylcytidine, and 1-methylguanosine. In some embodiments, the de-alkylating enzyme includes Escherichia coli (E. Coli) AlkB.

In various embodiments, the invention teaches a kit that includes a de-alkylating enzyme; and instructions for the use thereof to sequence an RNA. In certain embodiments, the RNA includes tRNA. In certain embodiments, the de-alkylating enzyme includes Escherichia coli (E. Coli) AlkB. In certain embodiments, the kit further includes one or more nucleotide primers or adapters specific for an RNA, and suitable for use in sequencing said RNA.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 depicts, in accordance with an embodiment of the invention, an ARM-Seq protocol schematic. AlkB-facilitated RNA methylated sequence (ARM-Seq) uses pre-treatment of RNA samples prior to RNA-seq library preparation to reveal RNAs containing AlkB substrates (m¹A, m³C, or m¹G). The workflow of commonly used protocols for obtaining small RNA sequencing reads (including NEBNext from New England Biolabs, and Illumina small RNA sequencing and TruSeq kits) requires ligation of sequencing adapters to the 3′ and 5′ ends of each RNA, prior to reverse transcription for library preparation and subsequent Illumina sequencing. In these “5′-dependent” cloning protocols, RNA modifications or secondary structures that block the progress of reverse transcription will produce cDNAs that lack the 5′ adapter sequence required for subsequent PCR amplification and sequencing. Without any additional treatments, the sequencing output from these protocols will therefore represent only those RNAs with appropriate end chemistry for the 5′ and 3′ sequencing adapter ligations (5′-monophosphate and 3′-OH, the expected end chemistry of mature tRNAs, some classes of tRNA-derived fragments, microRNAs, and snoRNAs) that do not contain impediments to reverse transcription. “Hard-stop” modifications such as m¹A, m³C or m¹G, which commonly occur in tRNAs, will cause premature termination of cDNA synthesis, preventing PCR amplification and subsequent sequencing. In ARM-Seq, demethylation with AlkB prior to library preparation facilitates sequencing of RNAs that contain m¹A, m³C, or m¹G, and comparative analysis of treated versus untreated samples provides a high-throughput profile of RNAs that contain AlkB-sensitive modifications.

FIGS. 2A-2D depict, in accordance with an embodiment of the invention, ARM-Seq reveals m¹A-modified tRNA fragments in S. cerevisiae. ARM-Seq increased the fraction of S. cerevisiae small RNA sequencing reads mapping to tRNAs by more than two-fold (A), with the majority of these corresponding to 3′-fragments and half-molecules of tRNAs where m¹A at T-loop position 58 (m¹A₅₈) is the most prevalent modification (B). ARM-Seq read profiles (C) show increases in 3′-fragment reads relative to untreated samples that predict the presence of m¹A₅₈ in Thr-AGT, Leu-GAG and Gln-TTG (each indicated by *). By contrast, ARM-Seq profiles for Arg-CCG, Gly-CCC and His-GTG show comparable or diminished reads over the T Loop region to untreated samples, predicting un-modified A₅₈ in these tRNAs. Primer extensions targeting the corresponding mature tRNAs (D) demonstrate that these ARM Seq results reflect the modification patterns of mature tRNAs, showing a hard stop that is removed by AlkB treatment at position 58 (indicated with an arrow for Thr-AGT), Leu-GAG and Gln-TTG tRNAs. These ARM-Seq and primer extension results confirm the A₅₈ modification state documented in Modomics for Thr-AGT and His-GTG, present corrective evidence that Gln-TTG contains m¹A₅₈ (in contrast to documentation in Modomics which shows un-modified A₅₈), and provide new information on the m¹A₅₈ modification state of Arg-CCG, Gly-CCC and Leu-GAG tRNAs.

FIGS. 3A-3C depict, in accordance with an embodiment of the invention, ARM-Seq predicts T-loop m¹A₅₈ modification state for S. cerevisiae tRNAs. ARM-seq log₂ fold changes reported by DESeq2 (A) show statistically significant increases of two-fold or more (indicated by the dashed red line, with P<0.01 indicated by *) for 22 of 26 S. cerevisiae tRNAs expected to contain m¹A₅₈ (85%) based on modifications documented in Modomics. In nearly all cases these corresponded primarily to increases in reads for 3′-fragments, indicating demethylation of m¹A₅₈ (B). Phe-GAA, Pro-TGG and Val-AAC-2 also showed increases in reads for 5′-fragment reads, consistent with demethylation of m¹G or other modifications. Fifteen of 19 tRNAs (79%) expected to contain un-modified A₅₈ showed no significant increase in ARM-Seq profiles compared to untreated controls (C), confirming documentation in Modomics; however, the remaining four in (C) with significant increases (*) indicate unknown presence of m¹A₅₈. Of the remaining nine tRNAs not represented in Modomics (D), five showed significant ARM-Seq responses (*) consistent with undocumented m¹A₅₈ modifications.

FIGS. 4A-4C depict, in accordance with an embodiment of the invention, ARM-Seq provides evidence for m¹A₅₈ modifications in the majority of human tRNAs and tRNA-derived small RNAs. ARM-Seq increased the proportion of small RNA sequencing reads mapping to tRNAs by approximately 3.5-fold in two B-cell derived human cell lines (A), with the increased reads in each case corresponding primarily to 3′-fragments and half-molecules where m¹A₅₈ is the most prevalent “hard-stop” modification (B). ARM-Seq responses for specific tRNAs were generally consistent between the two cell lines (Pearson correlation coefficient r=0.9), with increases of two-fold or more (dotted line) providing evidence for m¹A₅₈ modifications in the majority of human isodecoder groups (C). The left panel in (C) shows responses for tRNA subtypes with the lowest P-value or the highest ARM-Seq read count within each isodecoder group in lymphoma cells (GM05372). The right panel in (C) shows responses for the same subtypes in Epstein-Barr virus-transformed cells (GM12878). Significant responders are labeled (*).

FIGS. 5A-5D depict, in accordance with an embodiment of the invention, ARM-Seq profiles predict m¹A₅₈ modification state for human tRNAs. ARM-Seq profiles show significant increases consistent with m¹A₅₈ modifications for at least one subtype in 15 of 17 human isodecoder groups (88%) expected to contain m¹A₅₈ (A) and for 22 human isodecoder groups not currently represented in Modomics (B). Isodecoder subtypes with the lowest P-values were in many cases major subtypes that also showed the highest read count—profiles for both are shown where these differ. Primer extensions performed with or without AlkB treatment confirmed m¹A₅₈ modifications predicted by ARM-Seq for Pro and Cys tRNAs, which are not currently documented in Modomics for any mammal, and for Arg-ACG, where documentation is lacking for humans (C). ARM-Seq produced profiles consistent with documentation showing un-modified A₅₈ for several subtypes of Asp and Glu tRNAs (D), but also showed responses suggesting unexpected m¹A₅₈ modifications for Glu-CTC-1, Glu-TTC-1 and Glu-TTC-4. Primer extensions targeting the 3′-end of these Glu-CTC & Glu-TTC subtypes confirmed the presence of m¹A₅₈ (C). Control lanes in (C) show extensions using a primer for S. cerevisiae Thr-AGT in combination with S. cerevisiae (Y), human (H) or no RNA (−).

FIG. 6 depicts, in accordance with an embodiment of the invention, ARM-Seq provides evidence for early m¹A₅₈ modification of many human pre-tRNAs, and reveals m¹A, m¹G and m³C-modified mitochondrial tRNAs. ARM-Seq revealed modified RNAs derived from tRNA precursor transcripts, where the presence of 3′-trailers or 5′-leader sequences are distinguishing features not found in mature tRNAs (A). Read profiles for a subset of pre-tRNAs identified as significant provide evidence for modification of major and minor subtypes in a variety of isodecoder groups, including subtypes expected to contain m¹A₅₈ modifications based on modification patterns documented in Modomics, and others that are not represented in Modomics. Primer extensions performed with or without AlkB treatment confirmed the presence of an m¹A₅₈ modification in human Leu-CAA pre-tRNA (B). Primer extensions also show that AlkB treatment can demethylate m₁G as well as m₁A modifications in human mitochondrial tRNAs (C), confirming ARM-Seq results showing significantly increased reads for human mitochondrial tRNAs expected to contain m¹G or m¹A based on modification pattern documented in Modomics (c). ARM-Seq profiles for human mitochondrial tRNAs not represented in Modomics were often consistent with modifications documented for bovine mitochondrial tRNAs, and included significant responses for mito-Gln-TTG (where documentation for Bos taurus shows m¹G₃₇), mito-Glu-TTC (m¹A₉ & m¹A₅₈), mito-Ser-TGA (m³C₃₂ & m¹A₅₈), mito-Thr-TGT (m¹A₉ & m³C₃₂), and mito-Tyr-GTA (m¹G₉).

FIG. 7 depicts, in accordance with an embodiment of the invention, removing methylated nucleotides to sequence tRNAs. Whereas reverse transcriptase (RT) cannot extend through a fully modified tRNA (left), treatment with AlkB removes select methylated nucleotides to allow efficient reverse transcription and deep sequencing (center and right)

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

With the foregoing background in mind, in various embodiments, the invention describes RNA methylation sequencing which uses pre-treatment with a de-alkylating enzyme (e.g., Escherichia coli AlkB used in ARM-Seq) to demethylate 1-methyladenosine, 3-methylcytidine, and 1-methylguanosine, all commonly found in transfer RNAs (tRNAs). Comparative methylation analysis described in the “Examples” section using ARM-Seq provides the first detailed, transcriptome-scale map of these modifications, and reveals an abundance of previously undetected, methylated small RNAs derived from tRNAs. ARM-Seq demonstrates that tRNA-derived small RNAs accurately recapitulate the m¹A modification state for well-characterized yeast tRNAs, and generates new predictions for a large number of human tRNAs, including tRNA precursors and mitochondrial tRNAs. Thus, ARM-Seq provides broad utility for identifying previously overlooked methyl-modified RNAs, can efficiently monitor methylation state, and may reveal new roles for tRNA-derived RNAs as biomarkers or signaling molecules.

In various embodiments, the invention teaches a method that includes providing a ribonucleic acid (RNA); and applying a quantity of a de-alkylating enzyme to the RNA. In certain embodiments, the RNA includes all or a portion of a tRNA. In some embodiments, the RNA includes one or more of 1-methyladenosine, 3-methylcytidine, and 1-methylguanosine. In certain embodiments, the method includes sequencing all or a portion of the RNA, and thereby determining a post-de-alkylating-enzyme treated RNA sequence. In some embodiments, the de-alkylating enzyme may include, but is in no way limited to Escherichia coli (E. Coli) AlkB. In some embodiments, the ratio of de-alkylating-enzyme to RNA is 0.3 μg/1 μg-5 μg/1 μg, 0.5 μg/1 μg-4 μg/1 μg, 0.7 μg/1 μg-3 μg/1 μg, or 1 μg/1 μg-2 μg/1 μg. In some embodiments, the foregoing ratios are for E. coli AlkB/RNA. In some embodiments, the ratio (by weight) of E. Coli AlkB/RNA is 1:1.

With respect to treating RNA with AlkB, in some embodiments, a 200 μl reaction mixture containing 50 mM HEPES KOH, pH 8, 75 μM ferrous ammonium sulfate pH 5, 1 mM α-ketoglutarate, 2 mM sodium ascorbate, 50 μg/ml BSA, 50 μg AlkB, and 50 μg bulk RNA is incubated at 37° C. for one minute. In some embodiments, reactions are stopped by addition of 200 μl buffer containing 11 mM EDTA and 200 mM ammonium acetate. In some embodiments, the next steps are phenol extraction, ethanol precipitation, and resuspension of the washed pellet in water. In some embodiments, the ratio of the amount of AlkB to RNA can be any of the ratios described above. In some embodiments, functionally equivalent components of the reaction mixture can be substituted for those listed directly above. In certain embodiments, the incubation can be performed at a temperature ranging from 30 degrees or less to 45 degrees or more. In some embodiments, the duration of the incubation can be twenty seconds to two hours or more. In some embodiments, the pH can be 6-10, or 7-9, or 8. In some embodiments the concentrations of one or more components of the reaction mixture (or one or more functionally equivalent components) may be modified by 0.5-100%.

In some embodiments, the invention teaches a method that includes providing a sample that includes one or more nucleic acids (e.g., RNA or DNA), and applying a de-alkylating enzyme (including any de-alkylating enzyme described herein) to the sample, thereby forming a de-alkylating enzyme-treated sample. In some embodiments, the invention further includes sequencing (e.g., by any method described or referenced herein) all or a portion of a nucleic acid in the de-alkylating enzyme-treated sample, and identifying the presence or absence of one or more species of RNA or fragment thereof (including, but not limited to, a particular tRNA or fragment thereof) in the sample, based on the results of the sequencing (e.g., by comparative RNA analysis, as described herein). In some embodiments, the particular RNA species identified includes one or more methylated bases (e.g., any of the methylated bases described herein, such as, but not limited to, 1-methyladenosine, 3-methylcytidine, and 1-methylguanosine). In some embodiments, the sample is a biological sample (e.g., a biological fluid, biopsy, blood, tears, urine, etc.) obtained from a mammalian subject. In some embodiments, the mammalian subject is a human. In certain embodiments, the RNA species identified is associated with one or more disease condition in the subject. In some embodiments, the method further includes diagnosing an individual as having one or more disease condition on the basis of the sequencing results and the one or more RNA species (e.g., specific small RNA, tRNA, tRNA fragment, etc. containing one or more methylated base, as described above) that are detected. In some embodiments, the disease condition is a viral infection associated with one or more methylated bases (e.g., those described herein). In some embodiments, the viral infection is caused by the Epstein Barr virus. In some embodiments, the disease condition is hepatitis B or hepatitis C, and the tRNA species measured after AlkB treatment is a tRNA half, the increased presence of which is measured and associated with hepatitis B or hepatitis C (See Selitsky, S. R. et al. Small tRNA-derived RNAs are increased and more abundant than microRNAs in chronic hepatitis B and C. Scientific reports 5, 7675 (2015)). In certain embodiments, the disease condition is cancer which is associated with one or more methylated bases (e.g., those described herein). In some embodiments, the cancer is lymphoma. In some embodiments, the cancer is B-cell lymphoma.

In some embodiments, the invention teaches a method that includes, providing a sample that includes RNA, applying a de-alkylating enzyme to the sample (e.g. E. coli AlkB), thereby forming a demethylated sample, followed by 5′-independent library preparation, and comparative analysis of 5′-read end frequencies in the demethylated samples versus untreated controls. This method provides a high-throughput procedure to map methyl-modifications to specific nucleotide positions within modified RNAs (e.g., containing the methyl modifications described herein), as demonstrated in the “Examples” section.

In various embodiments, the invention teaches a composition that includes a ribonucleic acid (RNA) that has been treated with a de-alkylating enzyme. In some embodiments, the RNA includes tRNA, or a fragment thereof. In some embodiments, prior to treatment with a de-alkylating enzyme, the RNA included one or more of 1-methyladenosine, 3-methylcytidine, and 1-methylguanosine. In certain embodiments, the de-alkylating enzyme includes Escherichia coli (E. Coli) AlkB.

In various embodiments, the present invention provides a kit for RNA sequencing. The kit consists of or consists essentially of or comprises: a composition that includes a de-alkylating enzyme, including any de-alkylating enzyme described herein. In some embodiments, the kit further includes one or more components that may include, but are in no way limited to adaptors, nucleotides (e.g., fluorescently-labeled or non-fluorescently-labeled nucleotides), primers, enzymes, and buffers useful for RNA sequencing. In some embodiments, the components may include, but are not limited to, those utilized in next generation sequencing (e.g., single-molecule real-time sequencing (Pacific Biosciences), Ion semiconductor (Ion Torrent sequencing), Pyrosequencing (454), sequencing by synthesis (Illumina), sequencing by ligation (SOLiD), and chain termination sequencing, all of which are well-known in the art).

The exact nature of the components configured in the inventive kit depends on its intended purpose. In one embodiment, the kit is configured particularly for the purpose of sequencing RNA (including by high-throughput sequencing).

Instructions for use may be included in the kit. “Instructions for use” typically include a tangible expression describing the technique to be employed in using the components of the kit to affect a desired outcome. Optionally, the kit also contains other useful components, such as, containers, diluents, buffers, pipetting or measuring tools, or other useful paraphernalia as will be readily recognized by those of skill in the art.

The materials or components assembled in the kit can be provided in any convenient and suitable ways that preserve their operability and utility. For example the compositions can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

In various embodiments, the invention teaches a kit that includes a de-alkylating enzyme; and instructions for the use thereof to sequence an RNA. In some embodiments, the RNA includes tRNA or a portion thereof. In some embodiments, the de-alkylating enzyme includes Escherichia coli (E. coli) AlkB. In some embodiments, the kit further includes one or more nucleotide primers or adaptors suitable for use in sequencing said RNA by any method described or referenced herein.

Various embodiments of the present invention are described in the ensuing examples. The examples are intended to be illustrative and in no way restrictive.

### EXAMPLES

### Example 1

By way of additional background, next-generation RNA-sequencing has provided insight into the diversity and importance of small RNAs in a wide range of biological contexts. Fragments and half molecules derived from transfer RNAs (tRNAs) are often abundant constituents of small RNA sequencing libraries, and there is increasing evidence that these tRNA-derived RNAs can have important functions distinct from those of mature tRNAs, including potential roles in disease. However, tRNA-derived fragments are likely to escape detection by sequencing based methods when they contain nucleoside modifications similar to those in mature tRNAs. Many tRNA modifications are known to cause pauses or stops during reverse transcription, a critical step integral to most RNA-seq protocols. These so-called “hard-stop” modifications, including 1-methyladenosine (m¹A), 1-methylguanosine (m¹G), 2,2,-dimethylguanosine (m^{2,2}G), and 3-methylcytidine (m³C), are more prevalent in tRNAs than other classes of RNAs, and play critical roles in tRNA biogenesis, stability and function.

Although biochemical characterization studies show that these modifications block the progression of reverse transcriptase, several studies have documented nucleotide discrepancies in tRNA-derived halves and fragments, relative to the corresponding genes, at residues that are expected to be modified in mature tRNAs. This suggests that some reverse transcriptases used for RNA-seq library preparation may read through hard-stop modifications such as m¹A and m^{2,2}G at some low, unknown rate. However, it is not clear how frequently and in what context read-through occurs during cDNA synthesis from modified RNA templates, and thus modified RNAs cannot be reliably detected or quantitated based on nucleotide discrepancies in RNA-seq libraries.

Few studies have provided direct biochemical evidence that small RNAs derived from tRNAs contain post-transcriptional modifications similar to those of mature tRNAs, but it is likely that these modifications have important implications for the biogenesis, stability, and functional activities of tRNA-derived small RNAs, much as they do for mature tRNAs. For example, the presence of specific modifications can target specific tRNAs for cleavage into half-molecules, protect tRNAs from cleavage, or alter the interaction of tRNA fragments with proteins such as Dicer or Piwi.

Here, we describe an approach to improve the sensitivity of RNA-seq for detection of modified RNAs by pretreating RNA samples with a de-alkylating enzyme, Escherichia coli AlkB, prior to the reverse transcription step in library preparation. The known substrates of E. coli AlkB in RNA are m¹A, which is among the most common modifications in tRNAs (by one measure, approximately half of all tRNAs examined contained m¹A), and m³C, a less common modification also documented primarily in tRNAs. In each case, AlkB removes a methyl group to yield an unmodified residue (A or C). There is also evidence that E. coli AlkB can demethylate m¹G, a modification that is almost as prevalent as m¹A in tRNAs, although by a somewhat different mechanism.

Our analysis of samples from the model eukaryote Saccharomyces cerevisiae and from human cell lines shows that demethylation using AlkB produces striking changes in small RNA sequencing profiles. In particular, AlkB treatment greatly increases the abundance and diversity of reads for small RNAs derived from tRNAs, showing that most tRNA-derived fragments contain modifications found in corresponding mature tRNAs. This AlkB-facilitated RNA Methylation sequencing (ARM-seq) approach shows remarkable sensitivity and specificity, resolving m¹A modifications for tRNA-derived small RNAs that correctly matches the modification state of well-characterized S. cerevisiae tRNAs, Furthermore, ARM-Seq provides compelling evidence for m¹A modifications in a large proportion of human tRNAs where modification patterns were unknown or not well documented. Thus, ARM-seq facilitates sequencing of methyl-modified RNAs that otherwise escape detection in standard sequencing protocols, and can be used to characterize methylation patterns for large numbers of RNAs in parallel.

### Methods

Purification of E. coli AlkB

AlkB was purified after growth of 12 liters of E coli BL21(DE3) pLysS-bearing plasmid

JEE1167-B in the AVA421 vector, and induction with IPTG for 2 hours at 37° C. to express His6-3C-AlkB fusion protein. Crude lysates were made by sonication, and protein was purified by batch treatment on TALON resin, cleavage of the tag with His6-3C protease, re-application to TALON resin and retention of unbound protein, concentration of protein (Amicon Ultra-15 centifugal filter unit), gel filtration chromatography on a Hi-Load 16/60 Superdex 200 gel filtration column, and then storage of concentrated protein (15.4 mg/mL, 0.77 ml) in buffer containing 20 mM Tris-HCl pH 8.0, 50% glycerol, 0.2 M NaCl, and 2 mM dithiothreitol at −20° C., or at −80° C. Freezing did not impair activity.

### Growth of Yeast Cells and RNA Isolation

S. cerevisiae cells were grown in liquid YPD medium at 30° C. to OD⁶⁰°=1-2, and 300

OD-ml cells were harvested and quick frozen at −80° C. Then bulk RNA was prepared from cell pellets by the hot phenol method (see D'Silva, S., et al., A domain of the actin binding protein Abp140 is the yeast methyltransferase responsible for 3-methylcytidine modification in the tRNA anti-codon loop. RNA 17, 1100-1100 (2100)), typically yielding 2 mg of total RNA as measured with a Nanodrop spectrophotometer (Thermo Scientific, Waltham, Mass. USA). Total RNA samples from three independently inoculated cultures were each processed separately in subsequent treatments described below.

### Growth of Human Cell Lines and RNA Isolation

Cell pellets of the human B-lymphocyte derived cell lines GM05372 and GM12878 were purchased from Coriell Institute, Camden, N.J., USA and shipped frozen after PBS wash. Upon arrival, cells were immediately placed at −80° C. for storage prior to RNA extraction. Isolation of total RNA from 10⁸ human cells was performed using Direct-Zol™ RNA MiniPrep Kit (Zymo Research, Irvine, Calif., USA) with TRI Reagent (Molecular Research Center, Inc. Cincinnati, Ohio), typically yielding 400-450 μg of total RNA. Total RNA samples from each of the two human cell lines were then split into three technical replicates for subsequent treatments described below.

Treatment of RNA with AlkB

AlkB treatment of RNA was performed in a 200 μl reaction mixture containing 50 mM HEPES KOH, pH 8, 75 μM ferrous ammonium sulfate pH 5, 1 mM α-ketoglutarate, 2 mM sodium ascorbate, 50 μg/ml BSA, 50 μg AlkB, and 50 μg bulk RNA at 37° C. for one minute. Reactions were stopped by addition of 200 μl buffer containing 11 mM EDTA and 200 mM ammonium acetate, followed by phenol extraction, ethanol precipitation, and resuspension of the washed pellet in water. Control reactions for untreated samples were performed similarly, using AlkB storage buffer in place of AlkB enzyme.

### Primer Extension

For primer extension ˜0.7 pmol 5′-³²P-phosphorylated primer was annealed to 0.2 μg bulk RNA in 5 μl H₂O, by heating for 3 min at 95° C. followed by cooling to 50° C. and incubation for 1 h. The annealed primer was then extended using 64 U Superscript III 744 (invitrogen) in a 10 μL reaction containing first strand buffer (50 mM Tris-Hcl (pH 8.3, 25° C.), 75 mM KCl, 3 mM MgCl₂) and 1 mM of each dNTP for 1 h at 50° C., stopped by addition of 10 μl formamide loading dye and freezing on dry ice, and then primer extension products were resolved by electrophoresis on a 15% polyacrylamide gel containing 4 M urea, followed by visualization of the dried gel on a phosphoimager cassette.

### Size Selection and Preparation of RNA Sequencing Libraries

50 μg of control or AlkB treated RNA was processed using the MirVana miRNA Isolation Kit (Life Technologies Corporation, Carlsbad, Calif., USA) according to manufacturer's instructions to select for RNA <200 nt. The RNA was concentrated to 25 μg using RNA Clean and Concentrate-25 (Zymo Research, Irvine, Calif., USA), and 10 μg was treated with DNAse I (New England Biolabs, Incorporated, Ipswich, Mass., USA). Following column cleanup of the RNA, 1 ug was used as input for NEBNext small RNA library Prep Kit for Illumina (New England Biolabs, incorporated, Ipswich, Mass., USA).

Libraries were size selected on 2% SizeSelect agarose E-Gels, using the 50 bp E-gel ladder (Life Technologies Corporation, Carlsbad, Calif., USA) as a marker to select for bands corresponding to libraries from RNA between 18-120 nt. Dilutions from column cleaned and concentrated libraries were assessed by BioAnalyzer traces using Agilent High Sensitivity DNA kit (Agilent Technologies, Santa Clara, Calif., USA). Sequencing of the libraries was performed at the University of California, Davis DNA Technologies and Expression Analysis Core using Illumina MiSeq paired-end sequencing.

### Mapping of Sequence Reads

Reads were trimmed, removing barcoding indices and adapter sequences, and paired-end reads were merged using a custom python script (Seqprep, J. St. John), only merged reads corresponding to RNAs at least 15 nucleotides long were analyzed further. Reads were mapped to reference genomes (Homo sapiens 2009 assembly hg19, GRCh37 or S. cerevisiae April 2011 assembly sacCer3) plus the set of mature tRNA sequences from tRNAscan-SE tRNA gene predicitons for each of these genomes. Mature tRNA sequences were generated to account for post-transcriptional processing steps: predicted introns were removed, a CCA trinucleotide sequence was added to the 3′ends of all tRNAs, and a single G base was added to the 5′-end of His-GTG tRNA species. Each of these mature tRNA sequences were padded on both ends with 20 “N” bases to allow mapping of reads with additional end sequences. Reads were mapped to the reference genomes plus the non-redundant set of predicted mature tRNA sequences using bowtie2 (See Langmead, B. & Salzberg, S. L. fast gapped-read alignment with Bowtie 2. Nature methods 9, 357-359 (2012)) returning up to 100 alignments per read with default parameters. For analyses summarizing the composition of RNA-seq reads by RNA class, multiple mapping was not allowed and only the bowtie2 primary alignment was used (selected arbitrarily by bowtie2 when multiple features produced equal mapping scores). Each sample produced approximately one million mappable reads using this procedure. The proportional composition of these reads by RNA class was relatively uniform across technical replicates for the human samples, and somewhat more variable between biological replicates of the yeast samples that were derived from independently expanded cultures.

For differential expression analysis of individual genes and tRNAs using DESeq2 analyses (described below), all best matches according to the bowtie2 scoring function were used. Reads showing equal mapping scores to tRNA genes (which represent unprocessed pre-tRNA transcripts) or predicted mature tRNA sequences were mapped exclusively to mature tRNAs. Thus, reads with equivalent mapping scores to multiple gene loci (encoding identical mature tRNAs) were mapped instead to a single mature tRNA sequence. In addition, reads mapped by this procedure to tRNA gene loci all contain features of tRNA precursors that are not found in mature tRNAs (e.g. intronic sequences, 3′-trailers, or 5′-leaders). These pre-tRNA features often distinguish one tRNA gene locus from another even when the mature tRNA encoded is identical. Plots of read coverage profiles for tRNAs were produced using read counts that were normalized according to size factors calculated from DESeq2 analyses (see below).

### Differential Expression Analysis

Read counts were tabulated for all reads and assigned to mature tRNAs or genomic features where mapping produced at least 10 nucleotides of sequence overlap. Non-overlapping RNA sequences mapped to the same annotated genomic features were labeled and counted separately (for example non-overlapping RNAs mapped to a genomic feature annotated as HERVH-int were labeled HERVH-int.1, HERVH-int.2, . . . ) Read counts for all features that exceeded a minimum threshold of 20 reads were used as input to the DESeq2 R package with default parameters, as described in Love, M. I., Huber, W. & Anders, S. Moderated estimation of fold change and dispersion for RNA-seq data with DESeq2. Genome Biol 15, 550 (2014). DESeq2 takes into account variability between replicates, and normalizes read counts to account for differences in sequencing depth between samples, reporting ARM-Seq fold changes relative to untreated samples along with associated P-values that are adjusted for multiple hypothesis testing. The software pipeline developed for this study is available at http://lowelab<dot>ucsc<dot>edu/software/, and includes all necessary components for trimming raw sequence reads, merging paired-end reads, mapping reads, estimating abundance, making UCSC genome browser tracks, calculating differential expression, and generating RNA feature read coverage plots. All raw RNA-seq data have been deposited in the NCBI Short Read Archive under accession SRP056032.

New tRNA Naming Convention from RNA Central

tRNA transcripts and individual gene loci are labeled using a new systematic naming convention that is designed to be more stable and informative (Lowe and Chan, in preparation). The new tRNA naming convention echoes the systematic naming adopted for microRNAs in miRBase (See Griffiths-Jones, S., et al. miRBase: microRNA sequences, targets and gene nomenclature. Nucleic Acids Res 34, D140-144 (2006)). In brief, each unique mature tRNA transcript is named by isotype and codon (i.e. isodecoder), with each sequence subtype numbered in ascending order (e.g., tRNA-Ala-AGC-1, tRNA-Ala-AGC-2, etc.), from most “canonical” to least canonical (canonical is objectively defined by the bit score given to each tRNA by tRNAscan-SE using the default general tRNA model (See Lowe, T. M. & Eddy, S. R. tRNAscan-SE: a program for improved detection of transfer RNA genes in genomic sequence. Nucleic Acids Res 25, 955-964 (1997)). As with microRNAs, there are often multiple genome loci encoding identical mature tRNAs, so a secondary index number is assigned to denote specific tRNA gene loci (i.e. tRNA-Ala-AGC-1-1, tRNA-Ala-AGC-1-2, tRNA-Ala-AGC-1-3 describe different gene loci, but produce identical mature tRNA transcripts). Thus, labels for mature tRNA transcripts include only the first index number, which refers to the isodecoder subtype (e.g., tRNA-Ala-AGC-2), whereas labels for tRNA genes also include a second index, which refers to the locus number (i.e., tRNA-Ala-AGC-2-1). The new naming convention has been applied to all tRNAs in the Genomic tRNa database (See Chan, P. P. & Lowe, T. M. GtRNAdb: a database of transfer RNA genes detected in genomic sequence. Nucleic Acids Res 37, D93-97 (2009)), and has been adopted by the HUGO Gene Nomencalture Committee, and by RNAcentral (See The, R.C. RNAcentral: an international database of ncRNA sequences. Nucleic Acids Res 43, D123-129 (2015)). For convenience in cross-referencing, also include legacy labels from the genomic tRNA database, where tRNA genes were originally labeled by chromosome number and sequential order on chromosome (See Chan, P. P. & Lowe, T. M. GtRNAdb: a database of transfer RNA genes detected in genomic sequence. Nucleic Acids Res 37, D93-97 (2009)).

### Correspondence to Modifications Annotated in Modomics

Predicted mature tRNA sequences were compared to those from the Modomics database to annotate modifications. tRNAs were labeled with annotated modifications from Modomics when these contained matching anticodons and the sequence of originating (un-modified) bases in Modomics matched those of the genomically encoded tRNAss with three or fewer nucleotide mismatches. tRNAs that did not match Modomics tRNA sequences using these criteria were labeled as “not documented.”

Results—ARM-Seq Reveals an Abundance of Modified tRNA-Derived RNAs in the Model Eukaryote Saccharomyces cerevisiae.

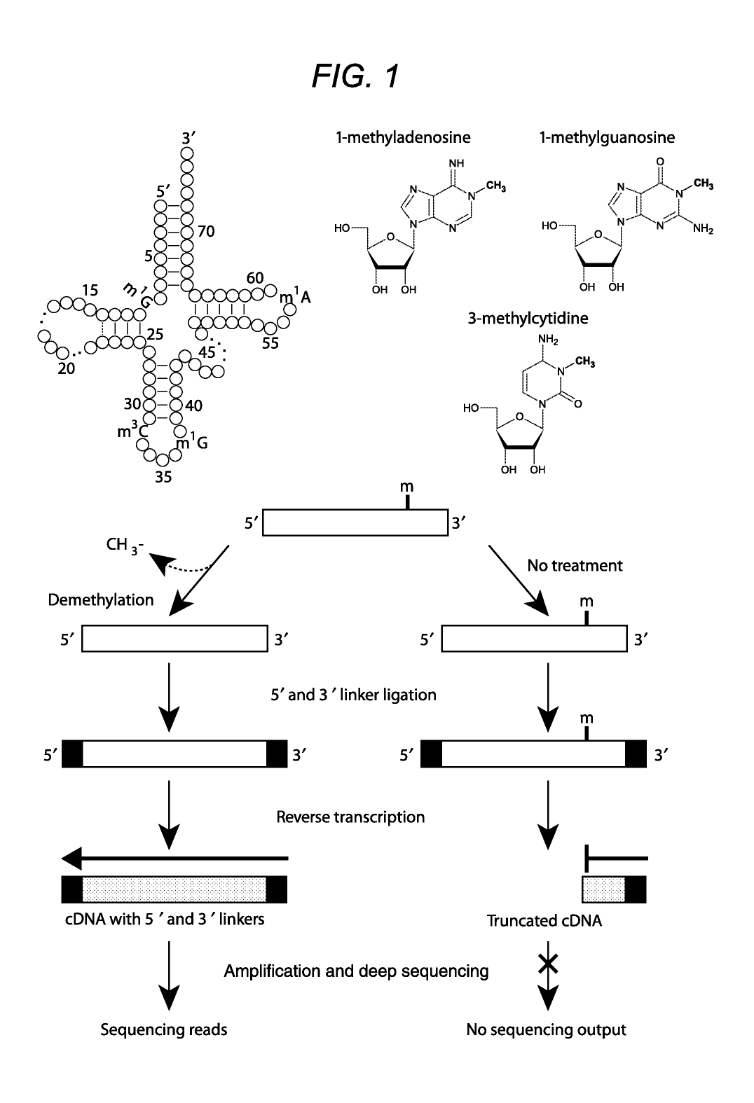

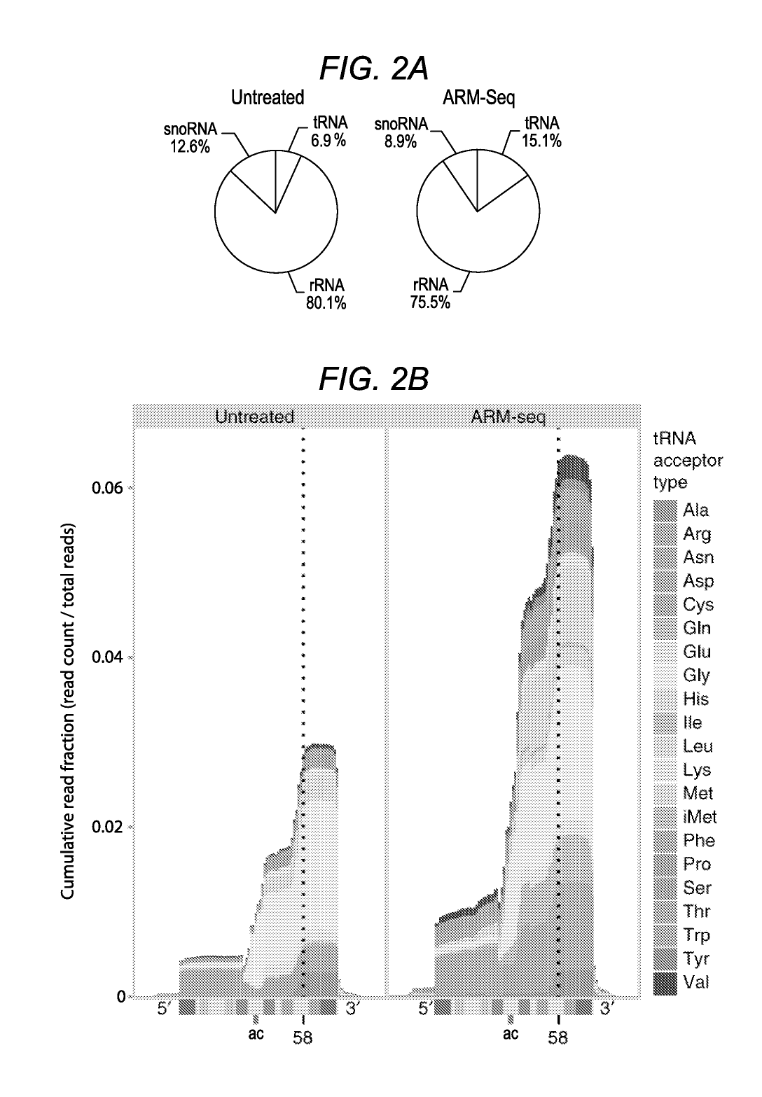

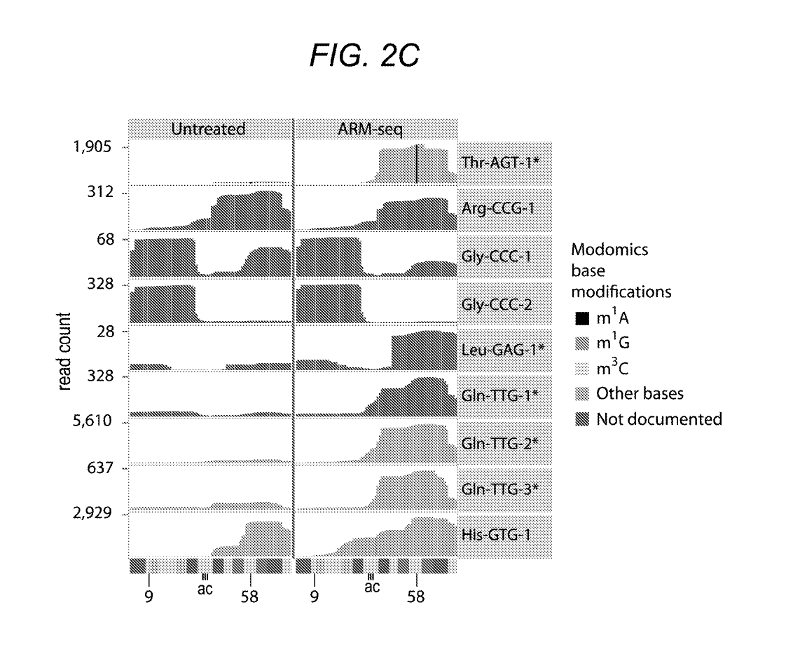

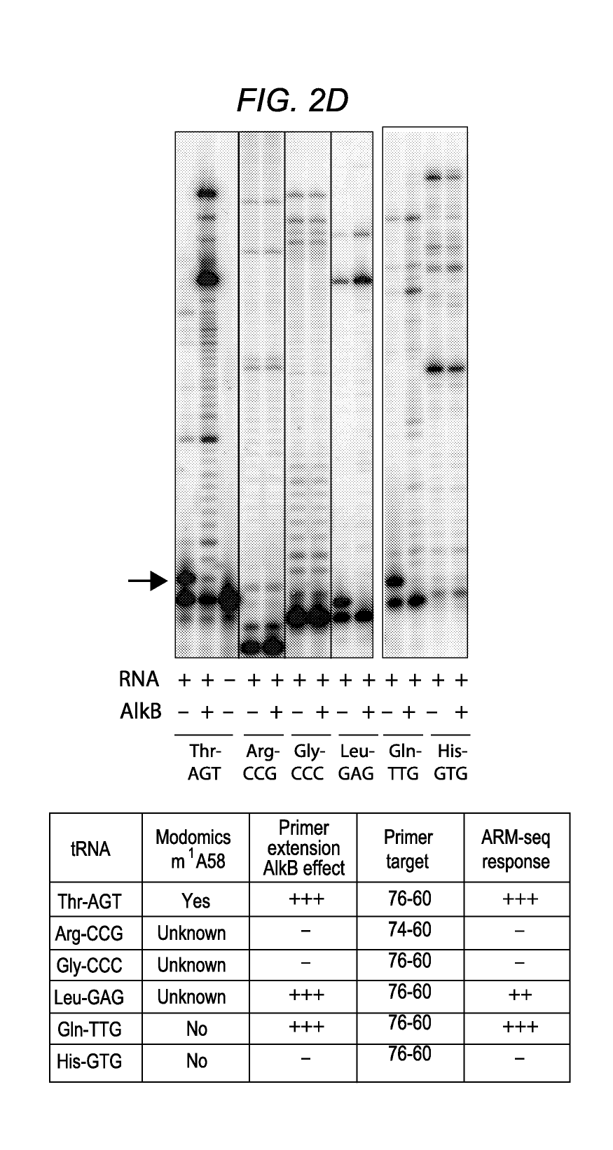

We first tested the ARM-seq methodology using small RNAS isolated from the budding yeast S. cerevisiae, where tRNA modifications have been extensively characterized through traditional biochemical analyses, and compiled in the comprehensive Modomics modification database (See Machnicka, M. A. et al. MODOMICS: a database of RNA modification pathways-2013 update Nucleic Acids Res 41, D262-267 (2013)). All sequencing was performed in triplicate using the NEBNext small RNA sequencing kit (New England Biolabs), which like many common RNA sequencing protocols is designed to capture substrates with 5′-monophosphate and 3′-OH ends that can be reverse transcribed into full-length cDNAs (see methods). RNAs containing nucleoside modifications or secondary structures that terminate reverse transcription prematurely cannot be amplified or sequenced, and are therefore absent in the sequencing output from these so-called “5′-dependent” cloning protocols (FIG. 1). In our experiments, application of ARM-Seq to S. cerevisiae samples more than doubled the proportion of small RNA sequencing reads from tRNA genes from 6.9% to 15.1% (FIG. 2a), providing evidence that these new fragments contain AlkB-sensitive modifications. In contrast, the proportion of reads mapping to other major classes of small RNAs (snoRNAs and rRNA fragments) diminished slightly (FIG. 2a). Precisely which portions of tRNAs were recovered from sequencing is an important dimension to our analyses. In our protocol, the small RNA size fraction selected for sequencing (<200 nt) is inclusive of mature tRNAs (typically −76 nt), yet reads for full-length mature tRNAs comprised less than 1% of the total read count for most tRNA types in both AlkB-treated and untreated samples. This result is consistent with an expected bias in sequencing library preparation in which the 5′ linker ligation is impeded by recessed 5′ ends of folded, full-length mature tRNAs. Instead, the gains produced by ARM-Seq reflect previously undetected, modified tRNA-derived fragments, which are the main focus of our study.

ARM-Seq Shows that the m¹A Modifications of tRNA-Derived Small RNAs Mirror Those of Mature tRNAs in S. cerevisiae

To further establish that AlkB treatment performs as expected when coupled to high throughput sequencing, we compared ARM-Seq read profiles to primer extensions targeting specific S. cerevisiae tRNAs. We focused in particular on the capacity to resolve m¹A modifications because most reads in both AlkB treated and untreated samples corresponded to 3′-fragments and half-molecules, where m¹A₅₈ is the most prevalent hard-stop modification (FIG. 2B). We first examined S. cerevisiae Thr-AGT tRNA, because it is known to contain m¹A₅₈. ARM-Seq produced an approximately 16-fold increase in normalized read count corresponding almost entirely to 3′-fragments and half-molecules that include A₅₈, consistent with AlkB-mediated demethylation of m¹A₅₈ in Thr-AGT derived small RNAs (FIG. 2C). Primer extensions using a primer targeting the 3′-end of mature Thr-AGT tRNA revealed a hard-stop band corresponding to m¹A₅₈ in an untreated sample, versus much reduced band intensity in the corresponding AlkB treated sample, consistent with demethylation of the expected m¹A₅₈ modification (FIG. 2D).

Similar comparisons for other S. cerevisiae tRNAs show that ARM-Seq consistently predicted the correct modification state of A₅₈ in mature tRNAs as verified by primer extension for both modified and unmodified tRNAs, His-GTG, a true negative for A₅₈ modification, was verified as unmodified (FIG. 2C,2D). Three tRNAs with no previous modification data were predicted as containing m¹A₅₈ (Leu-GAG) or having unmodified A₅₈ (Arg-CCG, Gly-CCC), and were then confirmed by primer extension (FIG. 2C, 2D). In addition, a tRNA type annotated as un-modified at A₅₈ (Gln-TTG) unexpectedly showed a strong increase in ARM-Seq indicative of m¹A₅₈ modification (FIG. 2C), which was clearly supported by primer extension data (FIG. 2d).

Comparative ARM-Seq Analysis Provides a High-Throughput, High-Resolution Assay for m¹A-Modified RNAS

Having demonstrated that ARM-Seq read profiles can predict m¹A₅₆ modification state for a small subset of S. cerevisiae tRNAs, we examined the effects of AlkB treatment for the complete set of S. cerevisiae tRNAs using DESeq2 (See Love, M. I., Huber, W. & Anders, S. Moderated estimation of fold change and dispersion for RNA-seq data with DESeq2. Genome Biol 15, 550 (2014)), a statistical method to assess significance of differential abundance of transcripts (see methods). Based on values derived from m¹A₅₈ true positives and negatives that were verified by primer extensions, we set a two-fold increase with a DESeq2 adjusted P-value <0.01 as our threshold for identifying significant changes in read abundance. A doubling of read counts in ARMseq versus untreated samples indicates the presence of AlkB-sensitive modifications in at least half of the detected RNA molecules derived from a given tRNA, while larger increases indicate an even greater proportion of modified molecules.

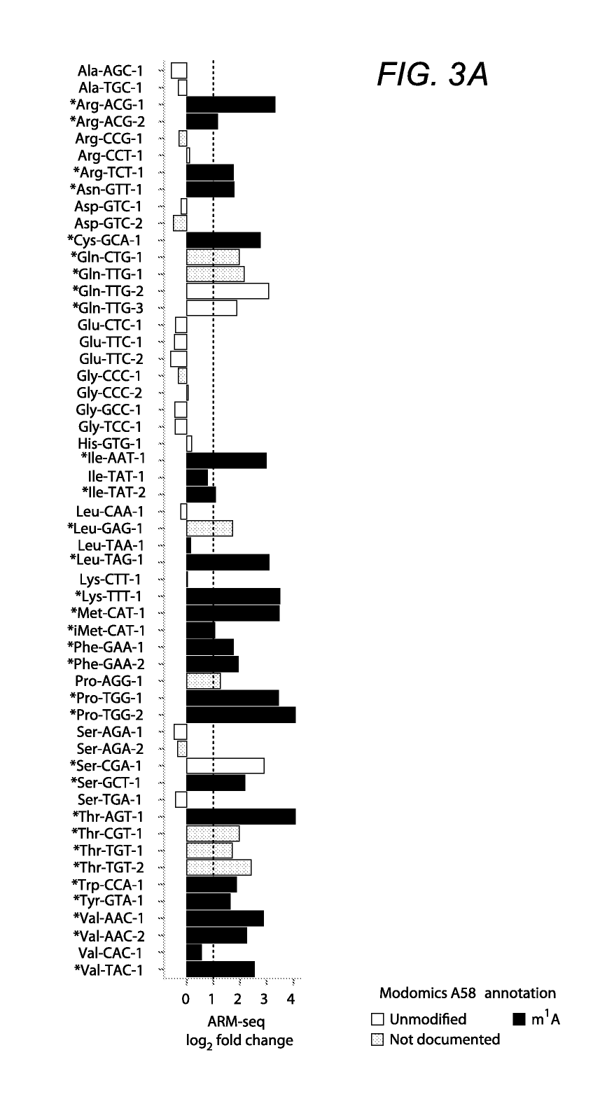

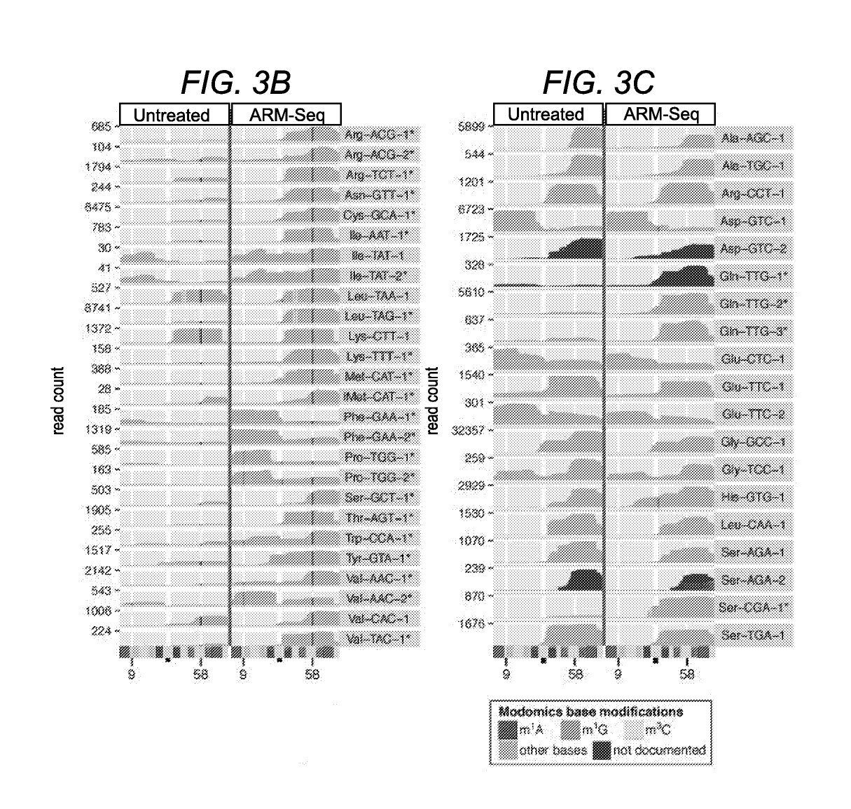

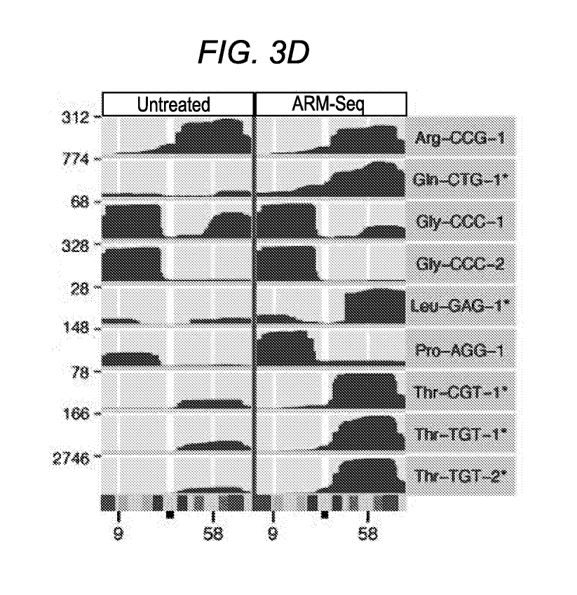

Overall, 56% of all cytosolic tRNAs in S. cerevisiae showed significant increases of two-fold or more in read count after AlkB treatment, a proportion that is roughly consistent with annotations showing m¹A modifications in 60% of the S. cerevisiae tRNAs documented in Modomics (FIG. 3A). Among the 26 specific S. cerevisiae tRNAs that were expected to contain m¹A modifications based on Modomics, 22 showed significant responses in ARM-Seq corresponding to increases in reads for 3′-fragments and 3′-half-molecules that included the A₅₈ position (FIG. 3A, 3B). Five of these 22 positives showed larger increases in reads for 5′-fragments that could be attributed to demethylation of m¹G₉ (for Pro-TGG-1, ProTGG-2, and Val-AAC-2) or possibly other 5′-domain modifications (for Phe-GAA-1, and Phe-GAA-2), but each of these also showed concomitant increases in 3′-fragment reads consistent with demethylation of m¹A₅₈ modifications (FIG. 3B). Of the 4 out of 26 remaining tRNAs expected to contain m¹A₅₈, two (Leu-TAA-1 & Lys-CTT-1) showed no increase in ARM-Seq consistent with unmodified A₅₈ residues, both of which were verified as unmodified by additional primer extension experiments. Taking into account these two primer extension verifications, we count 24 of 26 ARM-Seq predictions (92%) as correct. The two remaining positive tRNAs missed (Ile-TAT-1, Val-CAC-1) showed visible increases in reads for 3′-fragments and half-molecules (FIG. 3B), but these fell just short our thresholds for significance.

ARM-Seq results were also consistent with expectations for 15 of the 19 tRNAs in isodecoder groups expected to lack m¹A₅₈ based on Modomics-documented modification data. ARM-Seq profiles for these tRNAs showed comparable or diminished read counts for 3′-halves and fragments that included the A₅₈ position in AlkB treated samples compared to untreated samples, consistent with unmodified A₅₈ residues (FIG. 3A,3C). Exceptions that unexpectedly showed significant ARM-Seq responses included three Gln-TTG tRNAs, where m¹A₅₈ modification was confirmed by primer extension, as discussed above (FIG. 2D), yielding a successful prediction rate of 18 for 19 (95%). The one discordant exception was Ser-CGA, where Modomics documents an m³C modification at residue 32 in the anticodon loop, but not an m¹A₅₈ modification. Ser-CGA showed a ˜7.5-fold increase in reads for 3′-halves and fragments, but only half of these reads cover the documented m³C₃₂ position. Thus, the other half of the increased 3′-end reads provide evidence that the AlkB effect was due in part to demethylation of an undocumented m¹A₅₈.

Among the nine S. cerevisiae tRNAs in isodecoder groups not represented in Modomics, five showed significant ARM-Seq responses consistent with m¹A₅₈ modifications, including Leu-GAG-1 which was verified by primer extension (FIG. 3A, 3D, FIG. 2D). Three others showed ARM-Seq responses consistent with unmodified A₅₈, including the Arg-CCG and two Gly-CCC tRNAs, which were each verified by primer extension. The remaining tRNA not represented in Modomics, Pro-AGG, showed a 2.4-fold increase in ARM-Seq that was not quite significant (DESeq2 adjusted P value=0.011), with a large proportion of the increase corresponding to 5′-fragments, possibly indicating demethylation of m¹G₉ (FIG. 3A, 3D). Primer extensions targeting the 3′-end of Pro-AGG (not shown) also showed partial AlkB sensitivity, with incomplete removal of the block at m¹A₅₈ but an observable increase in read-through in AlkB-treated samples.

Thus, ARM-Seq reveals that small RNAs in S. cerevisiae include an abundance of m¹A-modified fragments derived from tRNAs. Moreover, ARM-Seq provides a high-throughput method to investigate m¹A₅₈ modification of tRNAs, facilitating transcriptome-scale assessment of modification patterns previously established through traditional, low-throughput biochemical analyses. In cases where ARM-Seq results disagree with prior modification data, or where there was no information for specific tRNAs, primer extensions are strongly supportive of ARM-Seq results.

ARM-Seq Shows that the Majority of tRNA-Derived Small RNAS are Modified in Human Cells

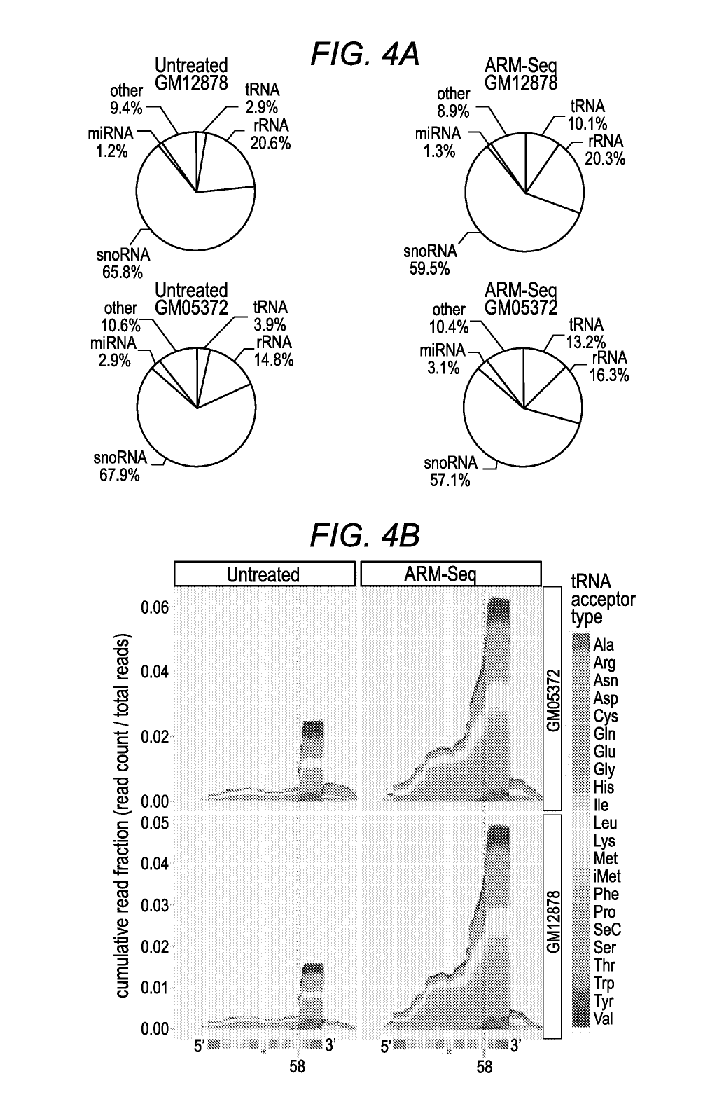

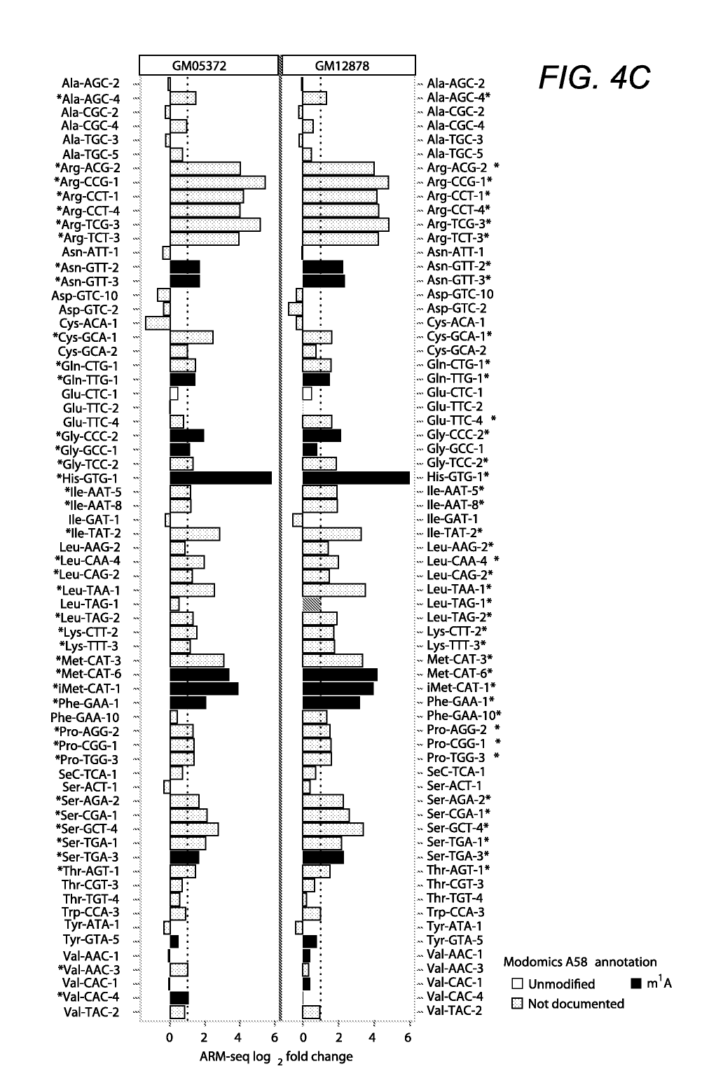

We next applied ARM-Seq to human samples, where the tRNA repertoire is more complex, and the details of tRNA processing and modification are much less well-characterized. Analysis of samples from two human cell lines revealed ARM-Seq responses even greater than those observed in S. cerevisiae. ARM-Seq increased the proportion of RNA-seq reads mapping to tRNAs from 2.9% to 10.1% in an Epstein-Barr virus transformed B-cell line (GM12878), and from 3.9% to 13.2% in a B-cell lymphoma derived cell line (GM05372), or about 3.5-fold in both cases (FIG. 4A). Most tRNA reads in the untreated human samples were short 3′-fragments beginning just downstream of the typically modified A₅₈ residue. ARM-Seq produced conspicuous increases in reads for 3′-fragments and 3′-half-molecules that include the A₅₈, consistent with demethylation of m¹A₅₈ (FIG. 4B). Although responses for specific tRNA species varied in strength between the two cell lines, corresponding tRNAs generally showed strongly correlated ARM-Seq responses in both sample types (Pearson r=0 9, FIG. 4c).

ARM-Seq Predicts m¹A₅₈ Modification State for Human tRNAs

Of 333 unique human tRNA gene sequences identified by tRNAscan-SE in the current reference genome (See Lowe, T. M. & Eddy, S. R. tRNAscan-SE: a program for improved detection of transfer RNA genes in genomic sequence. Nucleic Acids Res 25, 955-964 (1997) and Chan, P. P. & Lowe, T. M. GtRNAdb: a database of transfer RNA genes detected in genomic sequence. Nucleic Acids Res 37, D93-97 (2009)), just 43 match entries in Modomics, even allowing for up to three nucleotide differences in primary sequence (see methods), signifying the large amount of missing tRNA modification data relatable to the current draft of the human genome. Counted another way, Modomics currently includes modification patterns for 18 of 53 human cytosolic-type tRNA isodecoder groups. In contrast to yeast, m¹A₅₈ modification appears in nearly all of these human tRNA groups, 17 of 18, with Glu-CTC as the one exception.

ARM-seq positively predicted 15 of the 17 (88%) human isodecoder groups expected to contain m¹A₅₈ modifications, based on a significant response in at least one isodecoder subtype in the GM05372 B-cell lymphoma samples. A smaller subset of the same tRNAs (11 of 17 isodecoder groups, 65%) showed significant responses in the GM12878 samples, possibly indicating biological differences in tRNA fragmentation or modification patterns, a focus of ongoing studies (FIG. 4C). The remaining isodecoders expected to contain m¹A₅₈ (SeC-TCA and Tyr-GTA) showed increases in read count and proportional increases in read coverage of the T-loop, but did not meet the 2-fold increase threshold for significance. Based on the positions of tRNA fragment read count changes (FIG. 4D), ARM-Seq responses could be attributed in all cases to changes in m¹A₅₈ modifications.

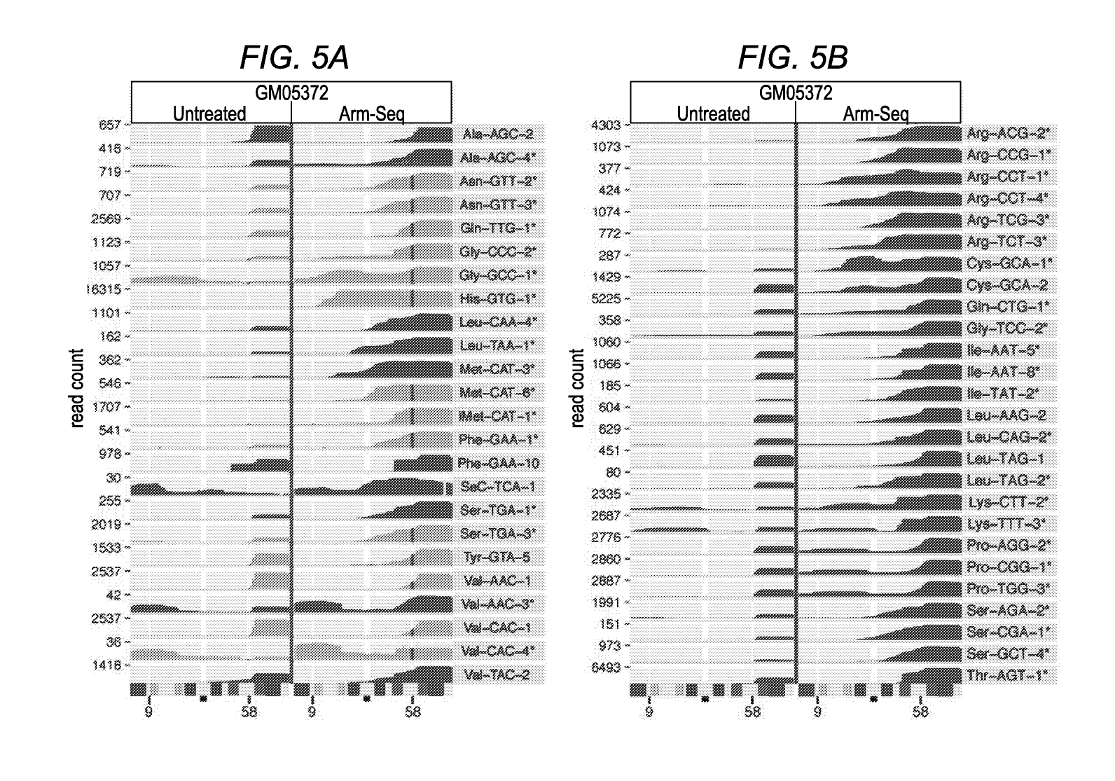

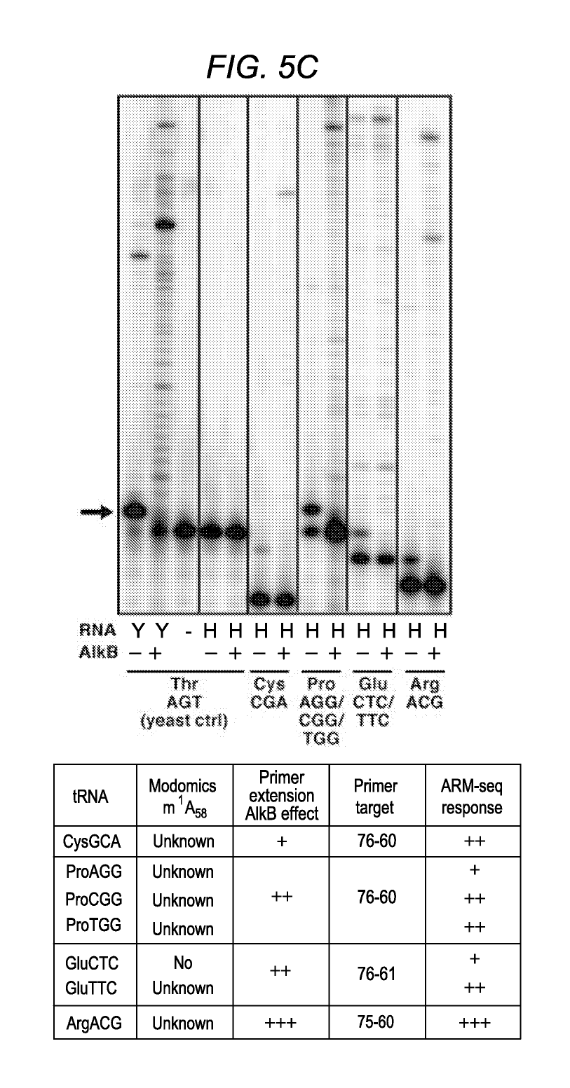

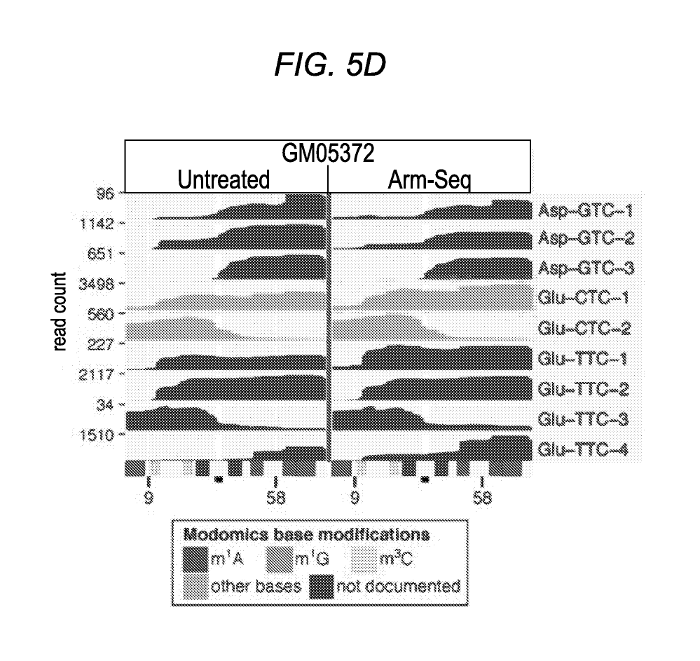

Among the 35 human isodecoder groups not currently represented in Modomics, ARM-Seq produced significant responses in 22 (63%) in the GM05372 samples, in each case corresponding to increases in 3′-fragment reads consistent with m¹A₅₈ modifications (FIG. 5B). Significant responders in the GM12878 samples included the same set, plus a subtype of Glu-TTC (67%). Primer extensions corroborated these results, providing evidence for m¹A₅₈ modifications for mature Cys and Pro tRNAs, which are not currently documented in Modomics for any mammal. Notably, m¹A₅₈ modification is documented in Modomics for Mus musculus Arg-ACG, and our results support this modification in previously undocumented human Arg-ACG tRNAs (FIG. 5C).

For the 13 human isodecoder groups not represented in Modomics and with non-significant ARM-Seq response, we either observed no reads covering the A₅₈ position, which neither confirms nor refutes the presence of m¹A₅₈ modifications, or we observed non-significant increases with only a portion of reads covering A₅₈, suggesting the presence of AlkB-sensitive m¹A modifications in a small fraction of the molecules detected. Indeed, Asp-GTC tRNAs and one subtype of Glu-TTC (Glu-TTC-2) showed decreases in normalized read count in AlkB-treated samples corresponding to tRNA 3′-halves and A₅₈-spanning fragments, indicating a lack of m¹A₅₈ modification in these tRNA-derived small RNAs (FIG. 5D). ARM-Seq results for human Asp-GTC are consistent with the modification pattern documented in Modomics for Asp-GTC in Rattus norvegicus, among the few mammalian tRNAs where Modomics shows A₅₈ as unmodified. In contrast, Modomics shows m¹A₅₈ modification in bovine Asp-GTC. ARM-Seq offers a means to further study the basis for these modification differences in the future.

Out of 18 human cytosolic tRNA isodecoders groups represented in Modomics, Glu-CTC is the only one documented with an unmodified A₅₈ residue. Human Glu-TTC is not represented in Modomics, but A₅₈ is also documented as unmodified in both Mus musculus Glu-CTC and Rattus norvegicus Glu-TTC. However, ARM-Seq and primer extension results together provide evidence for m¹A₅₈ modifications in human Glu-TTC-4 and possibly also Glu-CTC-1 and Glu-TTC-1 tRNA subtypes (FIG. 5C, 5D). Importantly, these findings indicate that the absence of m¹A₅₈ modifications documented in Modomics for human Glu tRNAs may apply only to specific isodecoder subtypes such as human Glu-TTC-2, where ARM-Seq results were consistent with unmodified A₅₈. Here, the individual tRNA resolution from ARM-Seq data have identified potentially important differences in modification among Glu-CTC and Glu-TTC tRNAs which merit follow-up study.

Overall, the remarkable agreement between traditional primer extension assays using total RNA (containing both full length and partial tRNAs) and ARM-Seq results (primarily capturing partial tRNAs) shows that the m¹A₅₈ modifications states for tRNA-derived small RNAs closely reflect those of mature tRNAs.

ARM-Seq Shows that Human Pre-tRNAs are m¹A Modified at an Early Stage of Processing

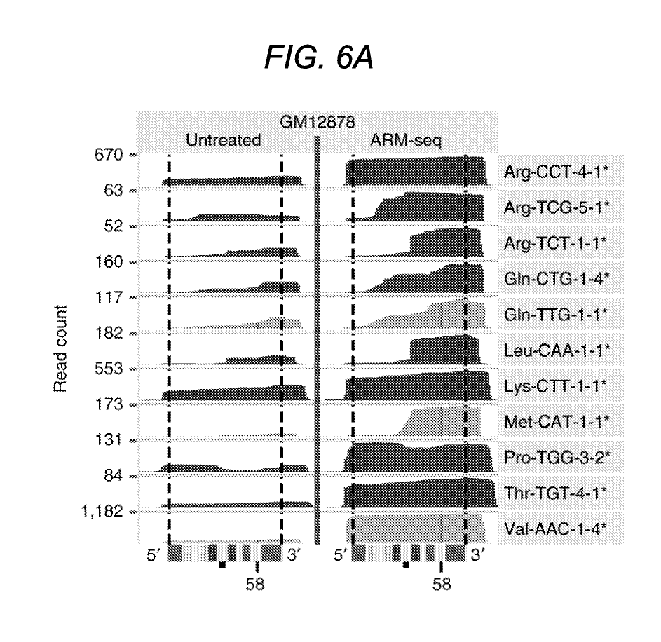

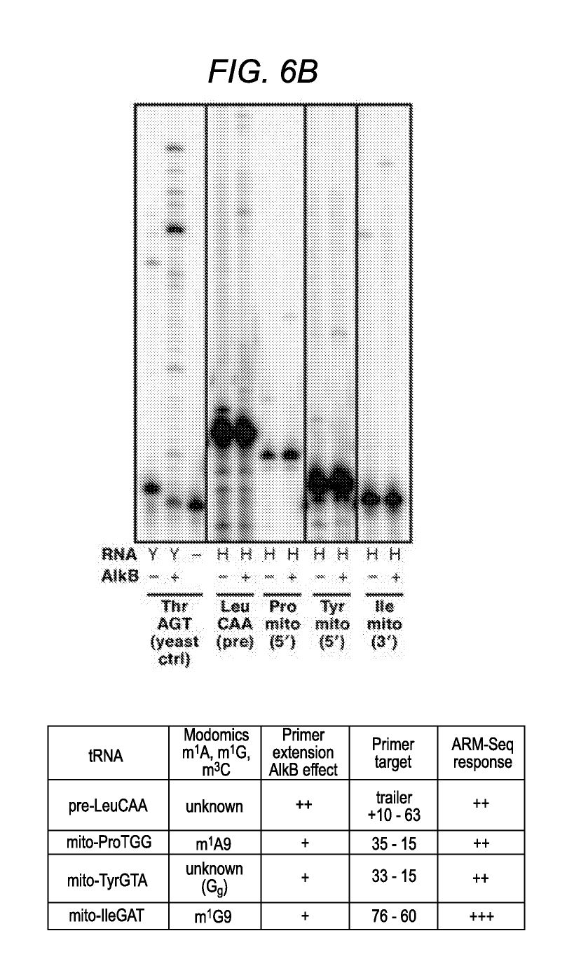

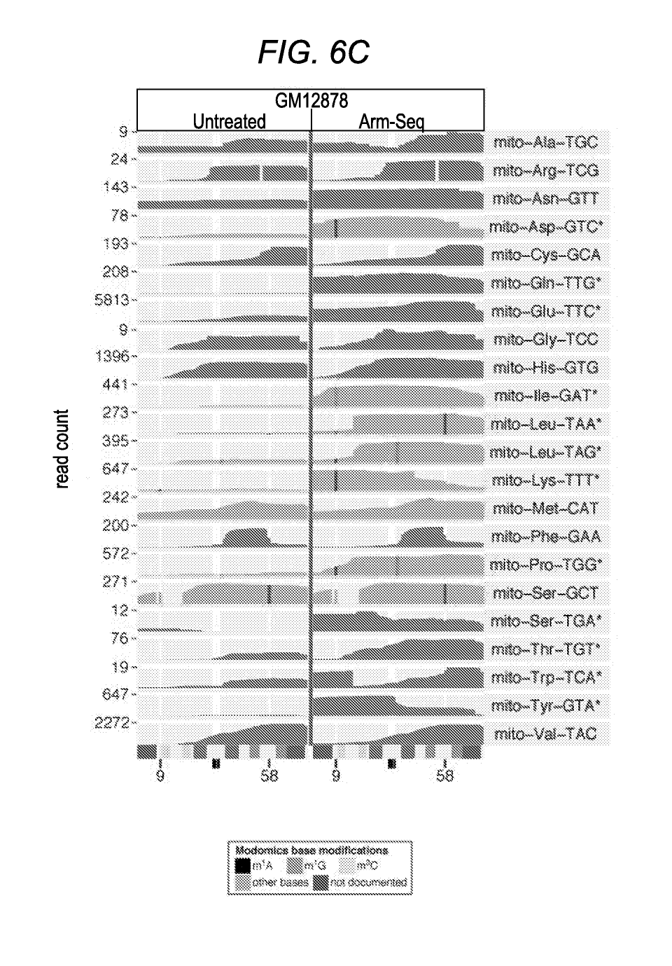

In contrast to the yeast samples, where AlkB treatment almost exclusively affected reads mapping to mature cytosolic-type tRNAs, the human samples also showed significant increases in reads that could instead be attributed to tRNA precursor transcripts. These reads preferentially mapped to tRNA genes rather than mature tRNAs because they included genomically-encoded sequences—most often 3′-trailer sequences but in some cases also 5′-leader sequences—that are found in tRNA precursor transcripts but not in mature tRNAs (FIG. 6A), The 5′-leader sequences of pre-tRNAs revealed by ARM-Seq were typically short (4-5 nt) when they were present, consistent with 5′-monophosphate ends (due to nucleolytic processing or dephosphorylation of triphosphorylated primary transcripts) required for RNA-seq library preparation. By contrast, the 3′-trailer sequences were typically 9-10 nt and sometimes longer, ending in many cases in a poly-T sequence, suggesting that these represent the 3′-ends of the primary RNA polymerase III transcripts. In each case, reads for full-length and fragmentary pre-tRNAs revealed by ARM-Seq included the T-loop region, which is consistent with m¹A₅₈ modifications. By contrast, isolated 3′-trailer fragments produced by 3′-end processing by RNaseZ (and which have been identified as associated with cell proliferation in several previous studies) showed a decrease in normalized abundance indicating that these do not contain AlkB-sensitive modifications. Although the presence of intronic sequences also distinguishes tRNA precursors from mature tRNAs, only a small fraction of reads included intronic sequences, and these showed little response in ARM-Seq (See Leu-CAA-1-1, FIG. 6A).

The processing steps that add nucleoside modifications to tRNAs are in many cases thought to occur after cleavage of 5′-leader and 3′-trailer sequences from tRNA-precursor transcripts. However, evidence demonstrating m¹A₅₈ modification of initiator methionine precursor transcripts in S. cerevisiae established a limited precedent for this particular modification at an earlier stage in pre-tRNA processing. Experiments demonstrating that S. cerevisiae Tyr-GTA precursors gain all T-loop modifications including m¹A₅₈ before subsequent processing when transcribed and processed in Xenopus laevis oocytes suggested that early m¹A₅₈ modification also occurs in higher eukaryote. However, direct evidence for m¹A₅₈ modification of endogenous pre-tRNAs for most organisms, including humans, has been lacking. ARM-Seq identified modified precursors for most human acceptor types, in each case producing increases in reads covering the A₅₈ position, consistent with m¹A₅₈ modifications. All together, pre-tRNAs in 33 different isodecoder families, and corresponding to 86 different human tRNA gene loci showed significant ARM-Seq responses in at least one of the two human cell lines analyzed. A large subset of these (28 different isodecoder families, corresponding to 38 different tRNA gene loci) showed significant ARM-Seq responses in both cell lines. It is noteworthy that these provide evidence for modified precursors of many major as well as minor human isodecoder subtypes, revealing expression and processing of specific tRNA genes that may be functionally distinct from others (e.g., see Arg-TCT-1-1, Arg-CCT-4-1, Pro-TGG-3-2, Thr-TGT-4-1 in FIG. 6A). Although pre-tRNAs are typically much less abundant and more challenging to detect than mature tRNAs, we were able to verify the presence of an m¹A₅₈ modification in a human Leu-CM pre-tRNA using primer extension (FIG. 6B). Thus, ARM-Seq provides the first evidence that many human pre-tRNAs are m¹A₅₈-modified prior to 5′-leader and 3′-trailer removal, which suggests that this pattern may occur broadly among eukaryotes.

ARM-Seq Reveals Modified RNAs Derived from Human Mitochondrial tRNAs

ARM-Seq also produced significant increases in reads mapping to human mitochondrial tRNAs, where the most frequent hard-stop modifications documented in Modomics are m¹A₉, m¹G₉, m¹G₃₇, and m¹A₅₈ (FIG. 6C). Modification patterns for only eight of 22 human mitochondrial tRNAs are currently documented in Modomics. Although modification patterns are documented for a complete set of bovine mitochondrial tRNAs, all except for initiator methionine show at least one difference in modification compared to the corresponding human tRNAs where both species are documented, underscoring the need for additional investigation to elucidate the modifications of human mitochondrial tRNAs, ARM-Seq revealed significant responses for 12 mitochondrial tRNAs in the GM12878 cell line, eight of which also showed significant responses in the GM05372 samples. In contrast to human cytosolic tRNAs, where ARM-Seq responses were attributable exclusively to m¹A₅₈ modification state, ARM-Seq profiles for human mitochondrial tRNAs provide evidence for demethylation of m¹A₉ (for mito-Asp-GTC, mito-Lys-TTT, and mito-Pro-TGG), m¹G₉ (for mito-Ile-GAT), and m¹G37 modifications (in mito-Leu-TAG and mito-Pro-TGG; FIG. 6c). ARM-Seq also produced a significant response consistent with an expected m¹A₅₈ modification for mito-Leu-TAA (although not for mito-Ser-GCT). Mito-Met-CAT, expected to contain no AlkB sensitive modifications, showed no change in ARM-Seq versus untreated samples. For human mitochondrial tRNAs not documented in Modomics, ARM-Seq profiles showed significant responses that in many cases were consistent with expected m¹G, m³C or m¹A modifications documented in Bos taurus mitochondrial tRNAs (FIG. 6C). Primer extensions confirmed AlkB-mediated demethylation of m¹A₉ for mito-Pro-TGG, and m¹G₉ in both mito-Ile-GAT and mito-Tyr-GTA (FIG. 6B).

### Discussion

The initial ARM-Seq results presented here show that a large fraction of small RNAs in both budding yeast and human cells contain base modifications that reflect biogenesis from modified mature tRNAs. Many of the RNAs revealed as highly abundant by ARM-Seq were nearly absent in untreated samples—fragments of Cys-GCA and Leu-TAG in S. cerevisiae and Arg-ACG and HiS-GTG in the human samples represent a few of many examples where this is true. Thus, comparative ARM-Seq analysis presents radically altered landscapes of tRNA fragments in two evolutionarily divergent model organisms. Recently developed protocols have provided tools to profile 6-methyladenosine (m⁶A), pseudouridine, and 5-methylcytidine (m⁵C) modified RNAs using high-throughput sequencing, in many cases revealing new and unexpected targets for these modifications. The ARM-Seq methodology adds the capacity to profile m¹A or m³C modified RNAs, which (unlike RNAs modified with m⁶A, pseudouridine, or m⁵C) are otherwise recalcitrant to sequencing, and likely to escape detection using standard RNA-Seq library preparation protocols. ARM-Seq also shows a somewhat unexpected capacity to reveal some m¹G-modified RNAs.

Comparative ARM-Seq analysis provides a high-throughput profile of m¹A₅₈ modifications that can be used to corroborate, extend, and in some cases correct tRNA modification patterns documented through traditional, low-throughput biochemical analyses. Furthermore, ARM-Seq results showing that many human pre-tRNAs are m¹A-modified demonstrate that ARM-Seq can provide important new insights into tRNA maturation that could help uncover modification-based regulatory checkpoints, Finally, ARM-Seq results revealing m¹A and m¹G-modified mitochondrial tRNAs suggest that this technology can be applied to investigate mitochondrial genetic diseases, where defects in mitochondrial tRNAs often play central roles. Our results (including both untreated and ARM-Seq samples) do not show the same evidence for nucleotide misincorporation at expected hard-stop modifications that has been reported in several other studies, suggesting that this phenomenon could be associated only with specific reverse transcriptases. Although such misincorporations are useful for identifying potentially modified residues, ARM-Seq is almost certainly more sensitive and quantitative for detection of modified RNAs because it does not depend on low-frequency aberrations in enzymatic behavior that are poorly understood, and possibly context-dependent. Importantly, the software pipeline developed with this method provides quantitative estimates for these modifications for any transcribed genomic feature, as well as highly informative, gene-specific read plot distributions that illustrate position-specific information.

In summary, the initial ARM-seq results presented here demonstrate capabilities that should facilitate the study of tRNA processing and modification in a wide range of biological settings, including investigation of novel model organisms, as well as comparative analyses of different developmental stages, tissue types, and disease states. Such studies may shed new light on the functions of tRNAs and tRNA-derived small RNAs, for example by revealing tissue-specific functions for distinct tRNA subtypes, or important regulatory functions for novel tRNA-derived small RNAs. It is noteworthy in this context that modified tRNA-derived RNAs outnumbered microRNAs by four-fold or more in the human cell lines analyzed here, which underscores their potential involvement in cellular signaling and regulation, as well as in pathogenesis of diseases such as cancer and viral infections (See Selitsky, S. R. et al. Small tRNA-derived RNAs are increased and more abundant than microRNAs in chronic hepatitis B and C. Scientific reports 5, 7675 (2015)), whether base modifications play central roles in these activities, and whether modifications have also obscured detection of other classes of RNAS, such as mRNAs or long non-coding RNAs, are among the many potential lines of research where ARM-Seq can be put to work going forward.

### Example 2

### Adaptation of ARM-Seq Procedures to Map the Nucleotide Positions of RNA Modifications Using 5′-Independent Sequencing Protocols

In the application of the ARM-seq procedure as described in Cozen et al (See Cozen A E, et al. 2015. ARM-seq: AlkB-facilitated RNA methylation sequencing reveals a complex landscape of modified tRNA fragments. Nature methods 12: 879-884.), RNA is demethylated prior to sequencing library preparation using standard, so-called “5′-dependent” cloning procedures designed specifically to clone full-length cDNAs derived from small RNAs. Standard procedures for small RNA sequencing are specifically designed to clone only full-length cDNAs derived from small RNAs in order to avoid simultaneously producing sequencing results from truncated cDNAs that represent only short 3′-segments of longer RNAs. This selectivity is typically achieved by requiring that all cDNAs contain the sequence of an adapter that is ligated to the 5′-end of RNAs prior to reverse transcription (the presence of the 5′-adapter sequence in cloned cDNAs is required for both PCR and sequencing steps subsequent to reverse transcription, hence the “5′-dependent” designation). Thus, untreated RNAs containing methyl-modifications that terminate reverse transcription prematurely produce truncated cDNAs lacking the 5′-adapter sequence required for sequencing, whereas these produce full length cDNAs that include the 5′-adapter sequence after demethylation treatment. The ARM-seq procedure as described can identify methyl-modified RNAs as those that are enriched in sequencing read abundance in libraries prepared from demethylated samples as compared to those prepared from untreated controls (See Cozen A E, et al. 2015. ARM-seq: AlkB-facilitated RNA methylation sequencing reveals a complex landscape of modified tRNA fragments. Nature methods 12: 879-884).

The basic ARM-seq procedure of a) demethylation pre-treatment, followed by b) sequencing and c) comparative bioinformatic analysis can also be adapted for use with so-called “5′-independent” protocols in order to both identify specific RNAs that are modified, and to pinpoint the specific nucleotide positions of modifications affected by demethylation treatment in a high-throughput manner. In the output from 5′-independent sequencing protocols, reads terminate more frequently at the positions of “hard-stop” methyl-modifications in untreated samples as compared to demethylated samples for reasons that are described as follows. In “5′-independent” sequencing protocols such as the TGIRT-based (Thermostable Group II Intron Reverse Transcriptase) procedure used by Zheng et al (See Zheng G, et. al. 2015. Efficient and quantitative high-throughput tRNA sequencing. Nature methods 12: 835-837.), adapter ligation to the 5′-ends of RNAs is not required, and both truncated and full-length cDNAs are cloned for sequencing. The sequencing output from “5′-independent” protocols is analogous to the output from a primer extension experiment, a well-established procedure in molecular biology in which a radiolabeled oligonucleotide primer is hybridized to specific target RNAs, followed by reverse transcription into cDNAs, and evaluation of the lengths of the resulting cDNAs using gel electrophoresis (See Carey et al. 2013. The primer extension assay. Cold Spring Harb Protoc 2013: 164-173.). In primer extension the 5′-ends of cDNAs can be used to identify the specific positions of “hard-stop” modifications in target RNA molecules, and the increased length of cDNAs can be used to verify removal of these modifications after demethylation treatment of RNA. Primer extensions were used in exactly this manner to demonstrate the demethylation activity of AlkB on target tRNA substrates with well-characterized modifications as a proof of principle for the ARM-seq procedure (See Cozen A E, et al. 2015. ARM-seq: AlkB-facilitated RNA methylation sequencing reveals a complex landscape of modified tRNA fragments. Nature methods 12: 879-884; Supplementary FIG. 51), and to verify new modifications predicted by ARM-seq results (See Cozen A E, et al. 2015. ARM-seq: AlkB-facilitated RNA methylation sequencing reveals a complex landscape of modified tRNA fragments. Nature methods 12: 879-884).

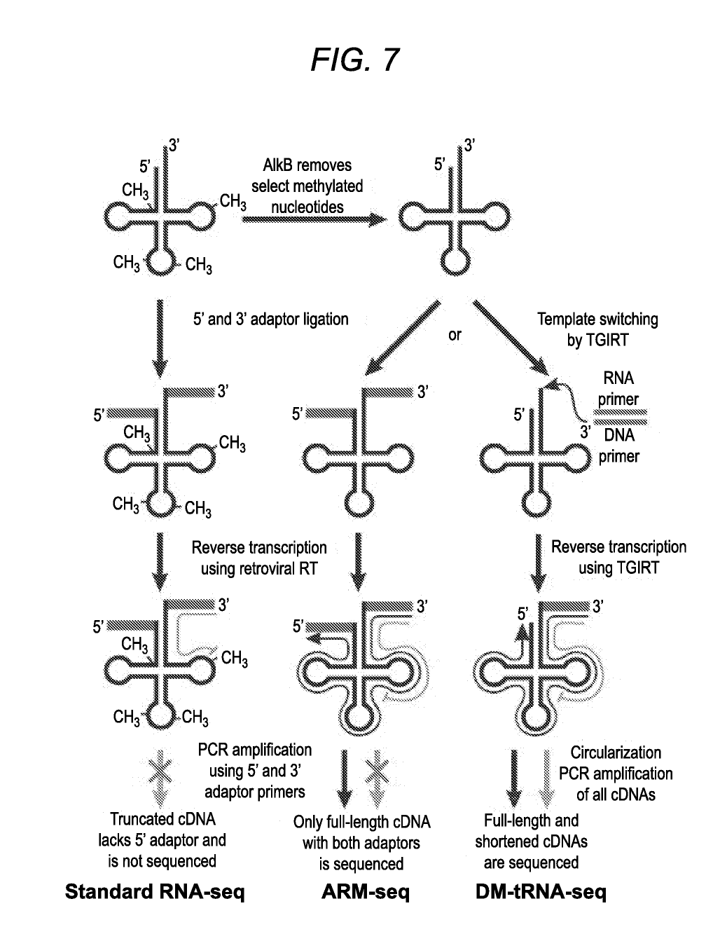

Zheng et al treated RNA with a mixture of wild-type E. coli AlkB plus mutant AlkB enzyme in order to facilitate production of longer cDNAs and sequencing reads derived from methyl-modified tRNAs using the 5′-independent TGIRT-based cloning procedure (See Zheng et al. 2015. Efficient and quantitative high-throughput tRNA sequencing. Nature methods 12: 835-837). Wilusz outlined key differences in the output from this procedure as compared to the 5′-dependent procedures described by Cozen et al (See Wilusz J E., et al. 2015. Removing roadblocks to deep sequencing of modified RNAs. Nature methods 12: 821-822 and FIG. 7 of the drawings submitted herewith). The increased proportion of cDNA products terminating at known hard-stop modifications in untreated controls compared to demethylated samples is shown for two tRNAs in Zheng et al FIGS. 2a & 2b (See Zheng G, et al. 2015. Efficient and quantitative high-throughput tRNA sequencing. Nature methods 12: 835-837). Whereas the procedure described by Zheng et al was designed to produce longer reads from modified tRNAs, the specific RNAs that were methylated and the exact positions of methyl-modifications were not analyzed in a high-throughput manner. ARM-seq bioinformatic procedures provide a work-flow for high-throughput analysis of such results, including position-specific evaluation in the context of modifications documented in the Modomics database (See Machnicka Mass., et al. 2013. MODOMICS: a database of RNA modification pathways—2013 update. Nucleic Acids Res 41: D262-267; and Cozen A E, et al. 2015. ARM-seq: AlkB-facilitated RNA methylation sequencing reveals a complex landscape of modified tRNA fragments. Nature methods 12: 879-884.). The essential adaptation in this case is the comparative analysis of 5′ read end frequencies in treated versus untreated samples for each RNA represented in sequencing results. Changes in 5′ read end frequencies that meet specified thresholds (e.g. a two-fold change as normalized to the total number of reads mapped to a given RNA) are used to identify the nucleotide positions of treatment-sensitive modifications and the RNA transcripts in which they occur. Thus demethylation pre-treatment, followed by 5′-independent library preparation, and comparative analysis of 5′-read end frequencies in demethylated samples versus untreated controls provides a high-throughput procedure to map methyl-modifications to specific nucleotide positions within modified RNAs.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method, comprising
providing a ribonucleic acid (RNA); and
applying a quantity of a de-alkylating enzyme to the RNA.

2. The method of claim 1, wherein the RNA comprises all or a portion of a tRNA.

3. The method of claim 1 wherein the RNA comprises one or more of 1-methyladenosine, 3-methylcytidine, and 1-methylguanosine.

4. The method of claim 2 wherein the RNA comprises one or more of 1-methyladenosine, 3-methylcytidine, and 1-methylguanosine.

5. The method of claim 1, further comprising sequencing all or a portion of the RNA, thereby determining a post-de-alkylating-enzyme treated RNA sequence.

6. The method of claim 1, wherein the de-alkylating enzyme comprises Escherichia coli (E. Coli) AlkB.

7. A composition, comprising a ribonucleic acid (RNA) that has been treated with a de-alkylating enzyme.

8. The composition of claim 7, wherein the RNA comprises tRNA.

9. The composition of claim 7, wherein the RNA comprised one or more of 1-methyladenosine, 3-methylcytidine, and 1-methylguanosine prior to treatment with the de-alkylating enzyme.

10. The composition of claim 8, wherein the RNA comprises one or more of 1-methyladenosine, 3-methylcytidine, and 1-methylguanosine.

11. The composition of claim 7, wherein the de-alkylating enzyme comprises Escherichia coli (E. Coli) AlkB.

12. The composition of claim 8, wherein the de-alkylating enzyme comprises Escherichia coli (E. Coli) AlkB.

13. A kit, comprising:
a de-alkylating enzyme; and
instructions for the use thereof to sequence an RNA.

14. The kit of claim 13, wherein the RNA comprises tRNA.

15. The kit of claim 13, wherein the de-alkylating enzyme comprises Escherichia coli (E. Coli) AlkB.

16. The kit of claim 14, wherein the de-alkylating enzyme comprises Escherichia coli (E. Coli) AlkB.

17. The kit of claim 13, further comprising one or more nucleotide primers specific for an RNA, and suitable for use in sequencing said RNA.
